(19) United States
(12) Patent Application Publication
(10) Pub. No.: US 2019/0224189 A1
(43) Pub. Date: Jul. 25, 2019

(54) USE OF TYROSINE-KINASE INHIBITOR IN PREPARING PHARMACEUTICAL PRODUCT FOR CANCER TREATMENT

(71) Applicant: Jiangsu Hengrui Medicine Co., Ltd., Lianyungang, Jiangsu (CN)

(72) Inventors: Jianjun ZOU, Lianyungang, Jiangsu (CN); Yaling HUANG, Lianyungang, Jiangsu (CN); Tonghuan HU, Lianyungang, Jiangsu (CN); Xiaoyu ZHU, Lianyungang, Jiangsu (CN); Jun FENG, Lianyungang, Jiangsu (CN); Changyong YANG, Lianyungang, Jiangsu (CN); Chen ZONG, Lianyungang, Jiangsu (CN); Guoqing CAO, Lianyungang, Jiangsu (CN)

(21) Appl. No.: 16/334,251

(22) PCT Filed: Sep. 22, 2017
(86) PCT No.: PCT/CN2017/102861

§ 371 Date: Mar. 18, 2019

(30) Foreign Application Priority Data
Sep. 23, 2016 (CN) 201610846757.1

(51) Int. Cl. A61K 31/4709; A61P 35/00

(52) U.S. Cl. CPC A61K 31/4709; A61P 35/00

(57) ABSTRACT

Provided is a method of treating cancer with a tyrosine-kinase inhibitor. In particular, provided is a method of treating cancer containing a mutated epidermal growth factor receptor (EGFR) with compound A, or a pharmaceutically acceptable salt thereof, preferably a maleate or dimaleate salt of compound A.

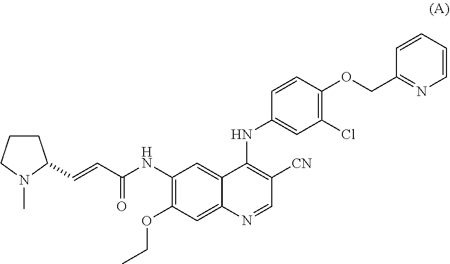

### FIELD OF THE INVENTION

Use of an EGFR/HER2 receptor tyrosine-kinase inhibitor in the preparation of a medicament for treating cancers with mutated EGFR.

### BACKGROUND OF THE INVENTION

Lung cancer has become a leading cause of deaths for both males and females with cancers in the world. According to the statistics of the Annual Report of Cancer Registration in China in 2015, both the incidence and mortality of lung cancer in China ranked in first place. The incidence and mortality of lung cancer increases with age. Generally, the incidence of lung cancer increases significantly after 40 years old, reaches peaks at about 75 years old, and then declines (Shi Yuankai, Sun Yan. Handbook of Clinical Oncology. Beijing: People's Medical Publishing House, 2015:315-341). In lung cancer, non-small cell lung cancer (NSCLC) accounts for approximately 85% of all patients with lung cancer (Siegel R, Ma J, Zou Z, et al. Cancer statistics. CA Cancer J Clin, 2014, 64(1):9-29). Adenocarcinoma is the most common pathological type of non-small cell lung cancer in developed countries, accounting for about 40%. Most patients already have NSCLC with local advanced or distant metastasis, which cannot be surgically removed, by the time they present.

The first-line treatment for metastatic NSCLC depends on the pathological types and genetic mutations. The treatment with EGFR-TKI is recommended for patients with positive EGFR mutations, and the administration of crizotinib is recommended for ALK-positive patients (Shi Yuankai, Sun Yan. Handbook of Clinical Oncology. Beijing: People's Medical Publishing House, 2015:315-341). EGFR gene-sensitive mutations include Exon19Exon19 Del, L858R and T790M, and the TKI medicaments against these mutations are erlotinib, gefitinib, icotinib, afatinib and osimertinib (AZD9291). Non-squamous carcinoma patients with negative expression of the above specific gene mutations are recommended to be treated with two-drug combination chemotherapy of pemetrexed or other platinum-containing drugs, and endostatin (YH-16) or cetuximab may be combined with the chemotherapy. Patients who have achieved control of the disease (complete remission, partial remission, or stable disease) after first-line treatment of 4-6 cycles may choose to continue maintenance treatment (given with at least one drug which has been administered in first-line treatment) or changing maintenance treatment (given with another drug which has not been administered in first-line treatment). Currently, the medical evidence supports the drugs such as pemetrexed (non-squamous carcinoma), gemcitabine, bevacizumab and EGFR-TKI (erlotinib) for maintenance treatment. For second-line treatment, docetaxel, pemetrexed and EGFR-TKI are available. There is no clear recommended therapeutic regimen for third-line treatment, and a large number of clinical trials are exploring other effective treatments (Shi Yuankai, Sun Yan. Handbook of Clinical Oncology. Beijing: People's Medical Publishing House, 2015:315-341). In addition to the above-mentioned targets, the studies on lung cancer have also revealed a number of abnormal alterations of genes, including amplification of MET and FGFR1, insertion mutation of EGFR exon 20 (Exon 20 ins), mutation of PIK3CA, AKT, KRAS, NRAS, BRAF, MEK1, AKT1, FGFR2, DDR2 and HER2, and rearrangement of RET and ROS1 (Mazières J, Peters S, Lepage B, et al. Lung cancer that harbors an HER2 mutation: Epidemiologic characteristics and therapeutic perspectives. J Clin Oncol, 2013, 31(16): 1997-2003).

Human epidermal factor receptor 1 (EGFR, HER1) gene is a member of the HER tyrosine receptor family. EGFR is a transmembrane receptor of glycoproteins that has tyrosine-kinase activity. Upon activation by binding to ligand, EGFR is converted from a monomer to a dimer. Such dimer may activate the autophosphorylation site of the EGFR intracellular domain, including activation sites such as Y992, Y1045, Y1068, Y1148 and Y1173, and guides downstream phosphorylation, including MAPK. Akt and JNK pathways, inducing cell proliferation, differentiation and cell survival. The EGFR mutation rate in NSCLC patients is 35% in Asia and 10% in the US (Lynch et al. 2004; Paez et al. 2004; Pao et al. 2004). 48% of these patients with EGFR mutations are Exon 19 Del (Mitsudomi and Yatabe 2010), 43% of them are L858R (Mitsudomi and Yatabe 2010), 4-9.2% of them are Exon 20 ins (insertion mutation in exon 20) (Arcila et al. 2013; Mitsudomi and Yatabe 2010; Oxnard et al. 2013), and <5% of them are T790M (Inukai et al. 2006). About 50% of patients with acquired resistance to erlotinib and gefitinib have T790M EGFR mutations (Kobayashi et al. 2005; Pao et al. 2005).

Currently, there is no specific drug targeting for EGFR Exon 20 ins. Preclinical studies indicate that these mutations are all resistant to gefitinib, erlotinib, neratinib and afatinib (Yasuda H 2012 Lancet Oncol.). In three retrospective analyses of Phase II and Phase III clinical studies for afatinib, 75 of the 600 patients (12%) with non-small cell lung cancer who received afatinib treatment had unusual mutations other than exon 19 Del and L858R, wherein, 23 patients had insertion mutation in exon 20, and ORR of the patients with Exon 20 ins evaluated through IRC was 8.7 (95% CI: 1.1%-28%); the median PFS was 2.7 months (95% CI: 1.8-4.2 months); and median OS was 9.2 months (95% CI: 4.1-14.2 months) (Yang et al. 2015 Lancet Oncol.). Naidoo et al found that 46 of the 1882 patients (2%) with stage IV lung adenocarcinoma carried Exon 20 ins, 11 of them received erlotinib treatment and 3 of the 11 patients (27%) were partial remission (PR), median TTP was 3 months (Naidoo 2015 Cancer). In summary, the benefits of NSCLC patients with Exon 20 ins from the treatment with commonly used EGFR TKI are very limited. Preclinical data showed that Ariad's drug under study AP32788 can effectively inhibit the enzymatic activity of some of EGFR Exon 20 ins proteins and the proliferation of some of these mutant cells (AACR 2016, François Gonzalvez, ARIAD Pharmaceuticals, Inc.), however, clinical trials are still ongoing and there is no published clinical trial data.

CN102471312B discloses a compound shown as the following formula A (chemical name (E)-N-[4-[[3-chloro-4-(2-pyridylmethoxy)phenyl]amino]-3-cyano-7-ethoxy-6-quinolyl]-3-[(2R)-1-methylpyrrolidin-2-yl]prop-2-enamide), and discloses that it has a strong inhibitory effect on EGFR and HER2 and is expected to be useful for the treatment of cancers overexpressing EGFR and HER2,

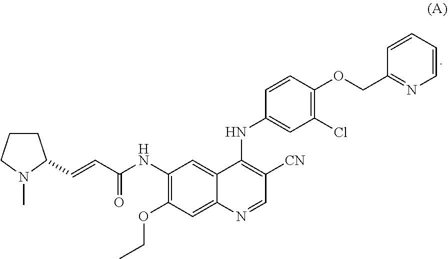

CN102933574B discloses a series of pharmaceutically acceptable salts of the compound of formula A. CN103974949B discloses a crystalline form of a dimaleate salt of the compound of formula A.

However, none of the above publications discloses the effect of the compound of formula A on the treatment of the cancers with EGFR mutations including L858R, exon 19 Del, T790M and exon 20 ins.

CN103987700A discloses an inhibitory effect of a class of tyrosine-kinase inhibitors on some EGFR mutant enzymes, however, the enzyme with exon 20 ins mutation is not included.

### SUMMARY OF THE INVENTION

The present invention has surprisingly found that compound A or a pharmaceutically acceptable salt thereof has an amazing effect on treating cancers with mutated EGFR, thereby the present invention is completed.

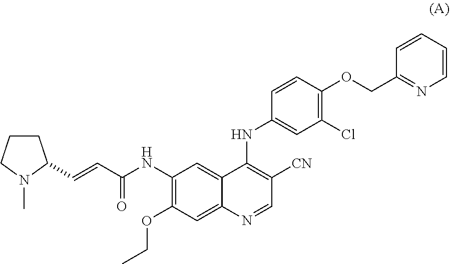

In a preferred embodiment of the present invention, the cancer is lung cancer, breast cancer, gastrointestinal cancer, kidney cancer, or liver cancer. Preferably, the lung cancer is non-small cell lung cancer, and further preferably non-small cell lung cancer with mutated EGFR, including adenocarcinoma, squamous carcinoma, large cell carcinoma, particularly, advanced non-small cell lung adenocarcinoma patients with mutated EGFR. Preferably, the gastrointestinal cancer is gastric cancer or colorectal cancer. Preferably, the breast cancer is a HER2-positive mutant breast cancer.

In the present invention, the tumor or cancer with mutated EGFR means that the EGFR driver mutation associated with cancer can be detected in these tumor or cancer patients, said mutation including but not limited to T790M, Exon19 Del, L858R and Exon 20 ins, preferably, the EGFR mutation described in the present invention is Exon 20 ins including but not limited to A763_Y764insFQEA. D770_N771insSVD, V769_D770insASV. H773_V774insNPH, H773_V774insH, H773_V774insPH, P772_H773insNP, D770_N771 insNPG, A763_Y764insFHEA, etc., wherein, T790M refers to the amino acid at position 790 being changed from T to M due to missense mutation of the base in the gene, Exon 19 Del refers to the non-frame-shift partial amino acid deletion due to the deletion of some base within exon 19, L858R refers to the amino acid at position 858 being changed from L to R due to a missense mutation of the base. A763_Y764insFQEA refers to the insertion of 12 bases in exon 20, resulting in the insertion of 4 amino acids FQEA between amino acid A at position 763 and amino acid Y at position 764. D770_N771insSVD refers to the insertion of 9 bases in exon 20, resulting in the insertion of 3 amino acids SVD between amino acid D at position 760 and amino acid N at position 771, V769_D770insASV refers to the insertion of 9 bases in exon 20, resulting in the insertion of 3 amino acids ASV between amino acid V at position 769 and amino acid D at position 770, H773_V774insNPH refers to the insertion of 9 bases in exon 20, resulting in the insertion of 3 amino acids NPH between amino acid H at position 773 and amino acid V at position 774, H773_V774insH refers to the insertion of 3 bases in exon 20, resulting in the insertion of 1 amino acid H between amino acid H at position 773 and amino acid V at position 774, H773_V774insPH refers to the insertion of 6 bases in exon 20, resulting in the insertion of two amino acids PH between amino acid H at position 773 and amino acid V at position 774, P772_H773insNP refers to the insertion of 6 bases in exon 20, resulting in the insertion of two amino acids NP between amino acid P at position 772 and amino acid H at position 773, D770_N771insNPG refers to the insertion of 9 bases in exon 20, resulting in the insertion of 3 amino acids NPG between amino acid D at position 770 and amino acid N at position 771. A763_Y764insFHEA refers to the insertion of 12 bases in exon 20, resulting in the insertion of 4 amino acids FHEA between amino acid A at position 763 and amino acid Y at position 764 (the position in amino acid sequence refers to P00533 EGFR_HUMAN).

In the present invention, the EGFR mutation includes not only the above single mutation type of the EGFR, but also a complex mutation type of any combination of T790M, Exon19 Del, L858R, and Exon 20 ins, including but not limited to T790M+Exon19 Del, T790M+L858R T790M+Exon 20 ins. Exon19 Del+L858R, Exon19 Del+Exon 20 ins, L858R+Exon 20 ins. In a preferred embodiment of the present invention, the cancer is a cancer that still progresses after chemotherapy, radiotherapy, targeted therapy or tumor immunotherapy. That is, patients with such cancer have ineffective treatment or are recurrent and progressive after chemotherapy, radiotherapy, targeted therapy or tumor immunotherapy, for example, the disease is not controlled and continues to progress, the tumor relapses and progresses after remission or stabilization. The chemotherapy described herein may be the treatment with various conventional chemotherapeutic drugs, including but not limited to alkylating agents (such as cyclophosphamide, isophosphamide, melphalan, busulfan, nimustine, ranimustine, dacarbazine, temozolomide, mechlorethamine hydrochloride, mitobronitol, etc.), platinum complexing agents (such as cisplatin, carboplatin, oxaliplatin, nedaplatin, etc.), metabolic antagonists (such as methotrexate, 5-fluorouracil, tegafur, gemcitabine, capecitabine, pemetrexed, anthracycline antibiotic, mitomycin, bleomycin, actinomycin, etc.), plant alkaloid such as vinblastine, camptothecin, taxanes, harringtonine (such as vincristine, catharanthine, vindesine, etoposide, docetaxel, taxoids, abraxane, paclitaxel liposome, irinotecan, vinorelbine, mitoxantrone, vinflunine, topotecan, etc.), hormone anticancer agents (such as leuprorelin, goserelin, dutasteride, fulvestrant, dexamethasone, tamoxifen, etc.), proteasome inhibitors (such as bortezomib, lenalidomide, etc.), aromatase inhibitors (such asexemestane, letrozole, anastrozole, etc.), preferably, chemotherapy is performed using one or more selected from the group consisting of carboplatin, cisplatin, oxaliplatin, 5-fluorouracil, Vinblastine, gemcitabine, camptothecin, antitumor antibiotics, endocrine inhibitor, pemetrexed or docetaxel. The targeted therapy can be the treatment using one or more selected from the group consisting of EGFR inhibitors, ALK inhibitors, PARP inhibitors, CDK inhibitors, MEK inhibitors, VEGF antibodies and VEGFR inhibitors. These targeted drugs are well known in the art, for example the EGFR inhibitors may be one or more selected from the group consisting of gefitinib, erlotinib, icotinib, afatinib, cetuximab, trastuzumab; the ALK inhibitor may be selected from the group consisting of crizotinib, ceritinib, axitinib, and brigatinib; the VEGF antibody is selected from bevacizumab; and the VEGF inhibitor is one or more selected from the group consisting of sunitinib, apatinib, famitinib. Tumor immunotherapy is one or more selected from the group consisting of nivolumab, pembrolizumab, atezolizumab and SHR-1210.

In the present invention, the compound A is preferably in the form of a pharmaceutically acceptable salt thereof in practical use, particularly in the form of a maleate or a dimaleate.

In the present invention, the daily dose of the compound A or a pharmaceutically acceptable salt thereof may range from 1 mg/kg to 20 mg/kg, preferably from 2 mg/kg to 10 mg/kg, from 10.1 to 14 mg/kg, from 14.1 to 18 mg/kg, from 18.1 to 20 mg/kg, more preferably from 4 to 8 mg/kg. For adults, the dosage range is preferably from 100 mg to 1000 mg, preferably from 240 mg to 560 mg, more preferably from 320 mg to 480 mg, based on the amount of compound A, wherein, for Asians, the daily dose may range from 240 mg to 400) mg, particularly 400 mg.

Compound A or a pharmaceutically acceptable salt thereof may also be formulated as the form of composition well known in the art with a pharmaceutically acceptable carrier, such as tablet, capsule, granule, injection and the like. The present invention also relates to the use of a pharmaceutical composition comprising compound A for the cancer with mutated EGFR.

The present invention also provides a method of treating the above-mentioned cancers with mutated EGFR, comprising administering compound A or a pharmaceutically acceptable salt thereof to a patient with EGFR mutated cancer.

### DETAILED DESCRIPTION OF THE INVENTION

The present invention is further described below in conjunction with the examples, but these examples are not intended to limit the scope of the present invention.

### Example 1

Effect of compound A and staurosporine on enzymatic activity of EGFR recombinant proteins (comprising insertion mutation in exon 20) in vitro.

### 1. Drugs to be Tested

Drug name: dimaleate of compound A (Lot Number: SHR 120201-002-06), staurosporine as control (MedChem MC-2104). Preparation method: all were prepared in DMSO.

### 2. Recombinant Protein

The ID number of EGFR gene is NM_0052288. The mutant proteins A763 Y764insFHEA (G1392-2, purchased from SignalChem) and D770_N771insNPG (G1368-2, purchased from SignalChem), that inserted into EGFR exon 20, are both the polypeptides from the amino acid at position 695 to the C-terminus of EGFR. Wild type EGFR protein (Cat# PV3872, Lot#39481M, purchased from Invitrogen) is the polypeptide from the amino acid at position 668 to the C-terminus of EGFR. These polypeptides are all expressed by baculovirus in Sf9 insect cells, and the N-terminus is labeled by GST.

### 3. Methods for the Test

The reaction system comprises 20 mM Hepes (pH7.5), 10 mM MgCl₂, 1 mM EGTA, 0.02% Brij35, 0.02 mg/ml BSA, 0.1 mM Na₃VO₄, 2 mM DTT, 1% DMSO and 10 μM ATP. BAS is a reaction substrate. The final concentrations of the enzymes for the reactions are: EGFR WT 4 nM, A763_Y764insFHEA 30 nM and D770_N771insNPG 15 nM. 10 μM-0.5 nM of the test compounds (compound A or staurosporine as control) were added into the reaction system, and then 0.01 μCi/μl of ³³P-ATP (Perkin Elmer) was added to initiate the phosphorylation reaction, so as to determine the enzymatic activity of EGFR. The statistical method is as follows: the abscissa corresponded to the logarithm of the concentration, and the ordinate corresponded to the probability unit of the inhibition rate at the corresponding concentration, and IC50 values were calculated using Prism4 software (GraphPad).

### 4. Results of the Test

The results showed that compound A had a strong inhibitory effect on the enzymatic activity of both recombinant human wild type EGFR and the mutated type with inserted mutation in exon 20, and as the concentration of compound A increased, the activity of EGFR holoenzyme gradually decreased, showing a concentration-dependent relationship. The results showed that compound A had a strong inhibitory effect on the enzymatic activity of both recombinant human wild type EGFR and the mutated type with inserted mutation in exon 20, however staurosporine had a poor effect, the specific results are shown in Table 1:

TABLE 1
Summary of IC₅₀ of compound A and staurosporine for
the in vitro enzymatic activity of EGFR recombinant proteins
(comprising insertion mutation in exon 20)
IC50 (nM)

| Recombinant Proteins | Compound A | staurosporine |
|---|---|---|
| EGFR WT | 0.545 | 173.3 |
| EGFR A763_Y764insFHEA | 0.148 | 349.8 |
| EGFR D770_N771insNPG | 0.435 | 105.2 |

### Example 2

Effect of compound A on in vitro enzymatic activity of EGFR recombinant proteins (comprising T790M. Exon19 Del and L858R).

### 1. Drugs to be Tested

Preparation method: all were prepared in DMSO.

| Compound | Lot Number: | Purity | Source |
|---|---|---|---|
| dimaleate of compound A | SHR120201-001-06 | 99.89% | JIANGSU HENGRUI |
| | | | MEDICINE CO., LTD |
| SHR141219 | SHR141219-002-06 | 97.64% | SHANGHAI HENGRUI |
| (BIBW-2992) | | | PHARMACEUTICAL CO., |
| | | | LTD |
| SHR118846 (HKI-272) | SHR118846-001-00 | 93.7% | SHANGHAI HENGRUI |
| | | | PHARMACEUTICAL CO., |
| | | | LTD |

### 2. Recombinant Protein

EGFR wild type (Cat# PV3872), EGFR T790M (Cat# PV4803). EGFR L858R (Cat# PV4128) and EGFR T790M+L858R (Cat# PV4879) recombinant proteins were all purchased from Invitrogen; EGFR Exon 19 Del (d746-750) (Cat#08-527) and EGFR Exon 19 Del+T790M (Cat#08-528) were purchased from Carna Biosciences. All of these recombinant proteins are the polypeptides from the amino acid at position 695 to the C-terminus.

### 3. Methods for the Test

IC50 detection was performed using the kit of Z′-LYTE™ Kinase Assay Kit-Tyrosine 4 Peptide (Invitrogen, Catalog No. PV3193). In the reaction system, the final concentration of DMSO was 2%, and the final concentration of the compound was in the range from 10,000 nM to 0.06 nM. The final concentration of the substrate was 2 μM. The final concentrations of the enzyme for reaction and the corresponding ATP were 0.58 ng/μL EGFR WT and 10 μM ATP: 1.5 ng/μL EGFR T790M and 10 μM ATP; 0.125 ng/μL EGFR T790M+L858R and 25 μM ATP: 0.75 ng/μL EGFR L858R and 50 μM ATP; 0.75 ng/μL EGFR Exon 19 Del and 40 μM ATP; 2 ng/μL EGFR Exon 19 Del+T790M and 10 μM ATP, respectively. The reaction was carried out according to the procedure described by the kit, and the fluorescence value was measured on a NOVOSTAR multi-function microplate reader. The statistical method is as follows: based on the fluorescence values, IC50 values were obtained from curve fitting of the inhibition rate to the log concentration of the compound by using GraphPad Prism 5 software.

### 4. Results of the Test

The results showed that both compound A and the positive control drug HKI-272 had similar inhibitory effects on EGFR wild-type and mutant lung adenocarcinoma cells (T790M. L858R and Exon19 Del, single mutation type and complex mutation type). The specific results are shown in Table 2:

TABLE 2
The inhibitory effect of compound A on in vitro enzymatic
activity of EGFR recombinant proteins (comprising T790M,
L858R and Exon19 Del, single mutation type and complex
mutation type).

| | | SHR141219 | SHR118846 |
|---|---|---|---|
| | Compound A | (BIBW-2992) | (HKI-272) |
| EGFR | IC₅₀ (nM) | IC₅₀ (nM) | IC₅₀ (nM) |
| EGFR WT | 42.31 | 3.31 | 32.47 |
| EGFR T790M | 135.00 | 7.24 | 81.46 |
| EGFR L858R | 25.14 | 1.11 | 20.10 |
| EGFR T790M + L858R | 242.20 | 10.89 | 197.60 |
| EGFR Exon19 Del | 125.70 | 3.77 | 72.95 |
| EGFR T790M + Exon19 | 1534.00 | 30.35 | 1711.00 |
| Del | | | |

1.-15. (canceled)

16. A method of treating cancer comprising mutated epidermal growth factor receptor (EGFR) in a subject in need thereof, the method comprising administering to the subject a pharmaceutical composition comprising a compound of formula A, or a pharmaceutically acceptable salt thereof, and a pharmaceutically acceptable carrier:

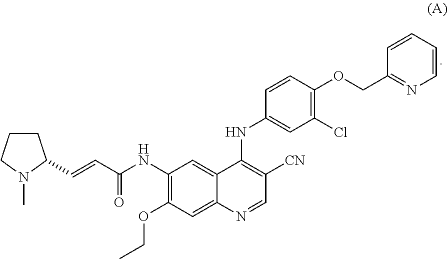

17. The method of claim 16, wherein the cancer is selected from the group consisting of lung cancer, breast cancer, gastrointestinal cancer, kidney cancer and liver cancer.

18. The method of claim 17, wherein the lung cancer is non-small cell lung cancer.

19. The method of claim 17, wherein the lung cancer is lung adenocarcinoma.

20. The method of claim 17, wherein the lung cancer is advanced lung cancer.

21. The method of claim 20, wherein the advanced lung cancer is recurrent refractory lung cancer.

22. The method of claim 16, wherein the mutation is a single mutation type or a complex mutation type selected from the group consisting of T790M, Exon19 Del, L858R and an insertion mutation in Exon 20 (Exon 20 ins).

23. The method of claim 22, wherein the mutation is Exon 20 ins.

24. The method of claim 16, wherein the cancer is a cancer which is still in progress or is recurrent and progressive after chemotherapy, radiotherapy or targeted therapy.

25. The method of claim 24, wherein the chemotherapy is performed by administering at least one chemotherapeutic agent selected from the group consisting of an alkylating agent, a platinum complexing agent, a metabolic antagonist, a plant alkaloid, a hormone anticancer agent, a proteasome inhibitor, an aromatase inhibitor and an immunomodulator.

26. The method of claim 25, wherein the chemotherapy is performed by administering at least one chemotherapeutic agent selected from the group consisting of carboplatin, cisplatin, oxaliplatin, 5-fluorouracil, vinblastine, gemcitabine, camptothecin, an antitumor antibiotic, an endocrine inhibitor, pemetrexed and docetaxel.

27. The method of claim 24, wherein the targeted therapy is a treatment comprising at least one selected from the group consisting of an EGFR inhibitor, PARP inhibitor, CDK inhibitor and VEGFR inhibitor.

28. The method of claim 27, wherein the EGFR inhibitor is at least one selected from the group consisting of gefitinib, erlotinib, icotinib and afatinib.

29. The method of claim 27, wherein the VEGFR inhibitor is at least one selected from the group consisting of sunitinib, apatinib and famitinib

30. The method of claim 16, wherein the pharmaceutically acceptable salt of compound A is maleate.

31. The method of claim 16, wherein the pharmaceutically acceptable salt of compound A is dimaleate.

32. The method of claim 16, wherein a daily dose of the compound A or the pharmaceutically acceptable salt thereof is 1 mg/kg to 20 mg/kg, based on the amount of compound A.

33. The method of claim 32, wherein the daily dose is 2 mg/kg to 10 mg/kg, based on the amount of compound A.

34. The method of claim 16, wherein the daily dose of the compound A or the pharmaceutically acceptable salt thereof is 100 mg to 1000 mg, based on the amount of compound A.

35. The method of claim 34, wherein the daily dose is 240 mg to 560 mg, based on the amount of compound A.
